(19) United States
(12) Patent Application Publication
(10) Pub. No.: US 2026/0334996 A1
(43) Pub. Date: Oct. 29, 2026

(54) Application cap of/for a plasma-surgical apparatus

(71) Applicant: Ovesco Endoscopy AG, Tübingen (DE)

(72) Inventors: Fabian SCHIMMELPFENNIG, Tübingen (DE); Gabor CONRAD, Tübingen (DE); Chi-Nghia HO, Reutlingen (DE); Marc SCHURR, Tübingen (DE)

(73) Assignee: Ovesco Endoscopy AG, Tübingen (DE)

(21) Appl. No.: 19/124,129

(22) PCT Filed: Oct. 25, 2023
(86) PCT No.: PCT/EP2023/079772

§ 371 Date: Apr. 24, 2025

(30) Foreign Application Priority Data
Oct. 26, 2022 (DE) 10 2022 128 370.4

(51) Int. Cl. A61B 18/04; A61B 17/00

(52) U.S. Cl. CPC A61B 18/042; A61B 2017/00473; A61B 2017/00738

(57) ABSTRACT

An application cap for/of a plasma-surgical instrument, provided as a spacer and designed to be coupled to/plugged on a distal end of an endoscopic supply device for the plasma-surgical instrument. The application cap is provided and designed to form a spatial volume at the distal portion thereof and to maintain at least a minimum distance between a distal end of an electrode insertable into the application cap and the target tissue and to have at least one wall region which is provided and designed to come into contact with a target tissue, and in which at least one treatment opening is formed. The at least one treatment opening forms a first treatment opening partial area and a second treatment opening partial area. The first treatment opening partial area is oriented in a different direction to the second treatment opening partial area.

### CROSS-REFERENCE TO RELATED APPLICATIONS

This application is a U.S. National Phase application of PCT International Application No. PCT/EP2023/079772, filed Oct. 25, 2023, which claims the benefit of DE 10 2022 128 370.4, filed Oct. 26, 2022, both of which are incorporated by reference herein.

### DESCRIPTION

### Technical Field

The present disclosure relates to an application cap for/of a plasma-surgical instrument, which is provided as a spacer, and to a plasma-surgical instrument for treating a target tissue with a plasma arc, comprising the application cap.

### Background to the Invention

Plasma surgical procedures as well as plasma applicators and similar devices/apparatuses for their application are generally known. For example, a plasma-surgical procedure known as fulguration or spray coagulation, in particular for thermal hemostasis, has been used in surgical operations for more than 50 years.

Plasma is generated exclusively in air, i.e. mainly oxygen plasma and nitrogen plasma are produced. Both plasmas are known to be chemically very reactive and generate carbonization effects, pyrolysis effects and consequently also vaporization of tissue and smoke on the tissue surface. These unintended side effects of fulguration or spray coagulation interfere with or hinder the application of this plasma-surgical procedure, in particular during endoscopic operations. Therefore, the gas was generally replaced by a noble gas, in particular argon, in order to avoid these disadvantages in the future.

### State of the Art

For example, DE 10 2008 004 843 B4 discloses a plasma treatment device which is used for flexible endoscopy. Due to its configuration, this apparatus can be used to perform a plasma-surgical treatment inside a human body. In the known surgical apparatus, a probe or catheter is laid inside an endoscope or is inserted into its working channel, which conducts the electrical energy from a high-frequency current/voltage source or from a generator to an electrode at the distal end of the probe/catheter (hereinafter referred to simply as the probe) via a connection line laid inside the probe/catheter.

The electrical energy applied to the electrode causes a plasma arc to form between the tip of the electrode and a body tissue/target tissue in the immediate vicinity. The electrode is located at the distal end of the probe and thus protrudes distally over the endoscope or out of its working channel.

According to DE 10 2008 004 843 B4, a resistive element, explicitly a capacitive resistive element, is additionally arranged between the distal end of the connection line laid in the probe and the proximal end of the electrode and is thus connected directly upstream of the electrode. The purpose of this resistive element is to limit the power supplied by the current/voltage source, hereinafter referred to as the ‘generator’, which flows through the target tissue and thus the human body. This arrangement ensures that the resistance element is placed as close as possible to the proximal end of the electrode. This proximity prevents the stray capacitances occurring in the system (due to conduction portions between the resistance element and the electrode) from having too great an influence on the voltage drop between the tip of the electrode and the target tissue. The current flowing through the target tissue is therefore mainly limited in a defined/definable manner by this resistor located between the connection line and the electrode.

### DESCRIPTION OF THE DISCLOSURE

The object of the present disclosure is to provide a spacer which enables safe and targeted treatment of target tissue. Furthermore, it is in particular an objective of the present disclosure to eliminate or at least improve disadvantages of the prior art.

This object is solved by an application cap/an attachment provided as a spacer and a plasma-surgical instrument.

Accordingly, the application cap/attachment for/of a plasma-surgical instrument, preferably of the minimally invasive type, is provided as an (electrically insulating) spacer and is configured to be coupled/plugged on/onto a distal end of an endoscopic supply device (endoscope) for the plasma-surgical instrument. The application cap is provided and configured to form a spatial volume (at least partially enclosed by a preferably rigid outer wall/circumferential wall of the application cap) at its distal portion, and to maintain at least a minimum distance of a distal end of an electrode insertable or introduced/inserted into the application cap from the target tissue. The application cap is further provided and configured to have at least one wall region, which in turn is provided and configured to come into contact with a target tissue and in which at least one treatment opening is formed. Here, the at least one treatment opening forms a first treatment-opening surface portion and a second treatment-opening surface portion, wherein the first treatment-opening surface portion points/is oriented in a different direction from the second treatment-opening surface portion.

In other words, the application cap has a wall region/body edge configured to come into contact with a target tissue and to maintain a minimum distance of the distal end of the electrode from the target tissue. Here, the application cap is provided at its distal portion in order to enclose a spatial volume and to form a treatment opening with a defined treatment-opening surface. The treatment-opening surface is positioned and/or oriented such that an electrode projecting into the spatial volume and in particular its distal end has a first predetermined minimum distance and a second predetermined minimum distance to the target tissue in the treatment-opening surface for generating the arc, wherein the treatment opening is provided and configured such that the wall region/the body edge rests proportionally on the target tissue such that either the first minimum distance or the second minimum distance is closer to the target tissue. In this case, the distance to the wall region/body edge does not change, but only by guiding the endoscope to the target tissue. This means that the distances of the treatment openings are different and therefore the distance is predefined and cannot be changed.

In other words, the application cap provides at least one treatment opening which extends over two, preferably adjacent sides (preferably in the radial/side direction and in the axial direction) of the application cap.

In other words, the basic idea of the present disclosure is to provide an application cap/spacer, preferably as a component/part separate from the feeding device/insertion aid/endoscope, which is adapted to be optionally mounted as an additional component to the distal end/end portion of the feeding device, for example of the endoscope type. The spacer/attachment/application cap initially has the function of holding the distal end of the feeding device at a certain distance from a patient-side target tissue when the spacer rests on the target tissue at a contact portion of the spacer provided for this purpose.

The application cap/attachment is configured in a cap-shaped manner such that a treatment surface/tissue contact surface of the application cap has a defined treatment opening/treatment-opening surface and a probe/electrode is arranged or can be inserted into the application cap/attachment. In more concrete terms, according to the invention, a type of attachment or an (application) cap for a feeding device (an endoscope) that can be plugged on or put on is proposed, with which a surgical instrument (electrode) can be introduced for the treatment of a target tissue/body tissue via a plasma arc, wherein the application cap/attachment is attached/attachable, in particular configured in the form of a cap, to the distal end/end portion of the feeding device or to its (endoscope) head. The electrode/probe has a distal end and a proximal end. The proximal end of the electrode is usually connected or connectable to an energy source. The energy source, alternatively also a generator or current/voltage source, provides the energy to form a plasma arc and thus enables the treatment of the target tissue.

The attachment is preferably rounded or convexly curved at least toward the outside. The attachment is made of an electrically non-conductive material, in other words an electrically insulating material, such as a plastic or a ceramic material.

Accordingly, the treatment opening in the application cap according to the invention has a portion/region that is configured/provided to rest on the target tissue/body tissue of a patient. The treatment opening or its border can therefore come into contact with the target tissue and thus define a treatment region which defines an area of a certain size corresponding to the tissue area intended for treatment. It is provided that the treatment opening is aligned both distally in the axial direction and to the side in the radial direction in such a way that contact with the target tissue is possible both distally and radially. At this point, it should also be pointed out that in the present case, it is always generally referred to a treatment opening aligned in two directions, wherein this can be understood to mean a single opening or at least two separate openings that are aligned in the two directions.

The application cap according to the invention has the advantage that, in the case of a single treatment opening, only one part, i.e. a first treatment-opening portion (in the distal direction) or a second treatment-opening surface portion (in the radial direction) of the treatment opening rests on/at the target tissue, depending on which treatment-opening (surface) portion causes the target tissue to be closer to the distal end of the electrode. It is therefore up to the user to guide the instrument with the application cap/attachment in such a way that a specific/desired part of the treatment opening or the treatment-opening surface lies on the target tissue. The option of choosing whether an arc is generated via the first or second treatment-opening surface portion makes it much easier for the user to reach the target tissue to be treated. Furthermore, placing only part of the treatment opening has the advantage that the positioning of a treatment-opening surface portion is further simplified for the user, since the attachment according to the invention offers more scope for exposing the target tissues.

Further aspects of the present disclosure according to the dependent claims are described below.

It is advantageous if the first treatment-opening surface portion and the second treatment-opening surface portion are configured contiguously so that they form a single treatment opening. In other words, the first treatment-opening surface portion together with the second treatment-opening surface portion form a single treatment opening which, however, extends over the sides of the application cap facing in different directions.

Alternatively, the first treatment-opening portion and the second treatment-opening portion may be configured to be separated from each other via a crosspiece, so that the first treatment-opening surface portion points in a first direction and the second treatment-opening surface portion points in a second direction, and the crosspiece forms a kind of edge between the two treatment-opening surface portions.

According to a preferred embodiment, the first treatment-opening surface portion and/or the second treatment-opening surface portion may be surrounded by a bearing edge/border configured to come into planar contact with the target tissue. Preferably, the bearing edge/border and the treatment-opening surface portion surrounded thereby may lie substantially in one plane. This ensures a flat bearing surface.

According to a further development of the preferred embodiment, a first bearing edge/border surrounding the first treatment-opening surface portion and the first treatment-opening surface portion may lie in a flattened/planar (bearing) region that extends transversely, preferably perpendicular, to the longitudinal axis of the application cap.

According to a further development of the preferred embodiment, a second bearing edge surrounding the second treatment-opening surface portion and the second treatment-opening surface portion may lie in a flattened/planar (bearing) region that extends off-center as well as at a tip angle inclined distally to the longitudinal axis of the application cap.

According to a further development of the preferred embodiment, the bearing edge/border may enclose the respective treatment-opening surface portion at least around half of the circumference of the respective treatment-opening surface portion or completely. This provides a suitably large surface for contact.

According to a preferred embodiment, the application cap may have a sleeve (enclosing a through hole) arranged on the side/laterally or a through hole arranged on the side/laterally, preferably with respect to the longitudinal axis of the application cap, through which the electrode can be inserted from the outside into the spatial volume of the application cap. This allows the electrode to be guided into the spatial volume.

According to a further development of the preferred embodiment, the application cap may be configured substantially closed on the outside, in particular on the circumferential side and/or on the end face, apart from the treatment opening and/or the sleeve or the through hole for the electrode. This ensures that the electrode only acts in the desired direction.

According to a further development of the preferred embodiment, the application cap may have a circumferential wall which encloses the spatial volume and in which the sleeve is arranged or in which the through hole is formed.

According to a preferred embodiment, the sleeve or the through hole may form a seal for sealing between the application cap and an electrode that can be inserted or plugged into the application cap through the sleeve or the through hole. This has the advantage that the electrode is sealed off from the outside.

According to a further development of the preferred embodiment, the seal may be formed as an elastic component that is separate from the circumferential wall, preferably inserted into the circumferential wall and bonded to the circumferential wall. For example, the seal may have a thermoplastic elastomer as material.

It is preferred if the application cap has the sleeve or the through hole, which is arranged and formed at a predetermined angle α, preferably between 20° and 90°, more preferably between 69° and 75°, and even more preferably (exactly) 72° inclined to the longitudinal axis of the application cap, in such a way that an arc can be generated in the direction toward the first treatment-opening surface portion or toward the second treatment-opening surface portion via the electrode which can be inserted into the application cap through the sleeve or the through hole, preferably depending on whether the target tissue is arranged closer to the electrode in the first treatment-opening surface portion or in the second treatment-opening surface portion. Alternatively or additionally, it is preferred if the predetermined angle α is between 20° and 45° when an arc is generated ‘forward’ in the frontal direction or substantially in the direction toward the first treatment-opening surface portion. Alternatively or additionally, it is preferred if the predetermined angle α is between 45° and 90° when an arc is generated ‘downward’ in the lateral direction or substantially in the direction toward the second treatment-opening surface portion.

It is advantageous if the first treatment-opening surface portion is arranged or formed at the distal end, preferably forming an orthogonal surface to the longitudinal axis of the application cap, and the second treatment-opening surface portion is arranged or configured substantially opposite the sleeve or the through hole.

It is advantageous if the second treatment-opening surface portion is inclined by more than 90° and less than 115°, preferably by 107.57° (according to (β) in FIG. 1) to the first treatment-opening surface portion. The angle of inclination of the second treatment-opening surface portion is inclined in such a way as to achieve optimum tightening/smoothing of the target tissues to be treated.

It is advantageous if the first treatment-opening surface portion has a first predetermined minimum distance to the electrode insertable through the sleeve and the second treatment-opening surface portion has a second predetermined minimum distance to the electrode insertable through the sleeve, wherein the first minimum distance is greater than the second minimum distance. Alternatively, it is preferred if the electrode is permanently installed in the sleeve and thus functions as a single unit.

It is preferred if the application cap is configured to fix the electrode to the outside of the application cap in a self-retaining manner while maintaining the predetermined first minimum distance and the predetermined second minimum distance. In other words, it is preferred if no working channel is provided through which the electrode/probe runs, but the electrode/probe can be fixed on the outside and can be inserted/pushed directly into the application cap via the sleeve. Alternatively, it is advantageous if the electrode can be inserted into a conventional working channel. This allows the tissue surface to be tightened and the distance to the tissue surface can be better estimated when being applied in a forward direction through the first treatment-opening surface portion.

It is advantageous if an edge is configured at the proximal end of the second treatment-opening surface portion and is provided for scraping or shaving off the target tissue. The edge is provided at the wall region, which is provided at the proximal end of the second treatment-opening surface portion. The edge preferably has a radius of less than or equal to 0.5 mm.

It is preferable if the sleeve is stretchable or flexibly configured at least when the electrode is inserted.

Furthermore, it is advantageous if the application cap is configured such that it can be plugged onto the plasma-surgical apparatus via an adapter. In other words, the application cap may integrally comprise an adapter at its proximal end which is provided and configured to be plugged on to the distal end of a plasma-surgical apparatus.

Alternatively, it is possible for the application cap and the adapter to be formed in two parts/separately from each other. In this case, the application cap and the adapter can be plugged together and can then be pushed onto the distal end of a plasma-surgical apparatus in combination.

It is advantageous if both the application cap and the adapter have a guide groove for guiding the electrode/probe.

The present disclosure further relates to a plasma-surgical instrument, preferably of the minimally invasive type, wherein an electrode is provided at the distal end of the instrument, adapted to generate an arc when supplied with electrical energy in a certain gas environment and at at least a predetermined minimum distance to a target tissue, with an application cap according to one of the preceding aspects.

### BRIEF DESCRIPTION OF THE FIGURES

FIG. 1 is a sectional view along a cross-sectional longitudinal axis A-A according to FIG. 3 of a first embodiment of the application cap according to the present disclosure;

FIG. 2 is a representation illustrating the first embodiment of the application cap according to the present disclosure;

FIG. 3 is a view from proximal toward distal of the application cap according to the present disclosure;

FIG. 4 is a bottom view of the application cap according to the present disclosure;

FIG. 5 is a side view of the application cap according to the present disclosure;

FIG. 6 is a top view of the application cap according to the present disclosure;

FIG. 7 is a schematic view of a plasma-surgical instrument with the application cap according to the present disclosure;

FIG. 8 is a schematic side view of a front part of the plasma-surgical instrument with the application cap according to the present disclosure;

FIG. 9 is an exploded view of the application cap, which can be plugged onto a plasma-surgical instrument;

FIG. 10 is an exploded view according to FIG. 9 from the side; and

FIG. 11 is a perspective view of a second embodiment of the application cap according to the present disclosure.

### DESCRIPTION OF THE CONFIGURATION EXAMPLES

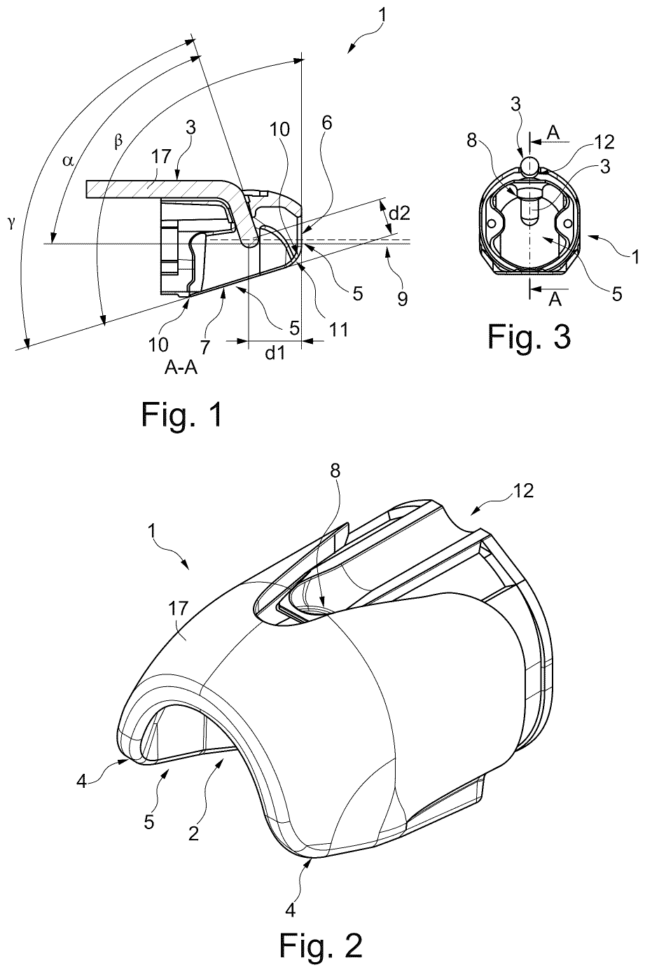

Hereinafter, an embodiment of the present disclosure will be described based on the accompanying Figures. FIGS. 1 to 10 show a first embodiment of an application cap 1 according to the present disclosure. FIG. 11 shows a second embodiment of the application cap 1 according to the present disclosure.

FIG. 1 is a sectional view along a cross-sectional longitudinal axis A-A according to FIG. 3 of the first embodiment of the application cap 1 according to the present disclosure. FIG. 1 shows the application cap 1 for a plasma-surgical instrument or of a plasma-surgical instrument, which is provided as a spacer. The application cap 1 can be plugged on or can be coupled to a distal end of an (endoscopic) supply device/endoscope. The application cap 1 forms a spatial volume 2 at its distal portion. In particular, the spatial volume 2 may be at least partially, preferably largely, enclosed by a preferably rigid outer wall/circumferential wall 17 of the application cap 1.

As shown in FIG. 1, the application cap 1 is provided and configured to house or insert an electrode 3, wherein it is provided to maintain at least a minimum distance d1, d2 from a distal end of the electrode 3 inserted into the application cap 1 to a target tissue.

The application cap 1 has a wall portion 4, which is provided and configured to come into contact with the target tissue. At least one treatment opening 5 is formed in the wall portion/region 4. The treatment opening 5 is an opening provided in the application cap 1 which reveals/exposes a region of the target tissue. The at least one treatment opening 5 forms a first treatment-opening surface portion 6 and a second treatment-opening surface portion 7. The first treatment-opening surface portion 6 and the second treatment-opening surface portion 7 are oriented in different directions.

In other words, the application cap 1 has, at a distal cap portion, substantially a hollow cone that is convexly rounded outward and whose cone tip and a circumferential wall portion are separated, resulting in a continuous opening which is oriented both distally in the axial direction (first treatment-opening surface portion 6) and laterally in the radial direction (second treatment-opening surface portion 7).

That is, as shown in FIG. 1, the first treatment-opening surface portion 6 faces in a distal direction and the second treatment-opening surface portion 7 faces in a radial direction (downward according to FIG. 1). The first treatment-opening surface portion 6 and the second treatment-opening surface portion 7 are, as already indicated, formed contiguously to form a single treatment opening 5. Alternatively, the first treatment-opening surface portion 6 and the second treatment-opening surface portion 7 may also be provided separated from each other by a crosspiece 11. In this case, the crosspiece 11 forms a kind of edge between the first treatment-opening surface portion 6 and the second treatment-opening surface portion 7.

The application cap 1 has a through hole 8 or a sleeve 8 enclosing the through hole, preferably radially opposite the second treatment-opening surface portion 7. The through hole or sleeve 8 is provided to house the electrode/probe 3 so that it protrudes into the spatial volume 2 of the application cap 1 in order to generate an arc with a target tissue in the direction of the first treatment-opening surface portion 6 or the second treatment-opening surface portion 7. The sleeve or the through hole 8 forms a seal for sealing between the application cap 1 and an electrode 3 that can be introduced or inserted into the application cap 1 through the sleeve 8 or the through hole 8.

The sleeve 8 or the through hole is provided at a predetermined angle to the longitudinal axis 9 of the application cap 1, so that an arc can be generated via the electrode 3 and a target tissue in the first treatment-opening surface portion 6 and the treatment-opening surface portion 7 without having to change the position of the electrode 3. The predetermined minimum distance d1 between electrode (tip) 3 and first treatment-opening surface portion 6 and d2 between electrode (tip) 3 and second treatment-opening surface portion 7 is maintained.

It is preferred if the predetermined angle α of the sleeve 8 to the longitudinal axis 9 is substantially between 10° and 80°, preferably 72°. Alternatively or additionally, it is preferred if the predetermined angle α is between 20° and 45° when an arc is generated ‘forward’ in the frontal direction or substantially in the direction toward the first treatment-opening surface portion 6. Alternatively or additionally, it is preferred if the predetermined angle α is between 45° and 90° when an arc is generated ‘downward’ in the lateral direction or substantially in the direction toward the second treatment-opening surface portion 7.

As shown in FIG. 1, the first treatment-opening surface portion 6 is arranged/configured at the distal end. This means that the first treatment-opening surface portion 6 forms an orthogonal surface to the longitudinal axis 9 of the application cap 1. The second treatment-opening surface portion 7 is arranged substantially opposite the sleeve 8. The second treatment-opening surface portion 7 is preferably inclined by more than 90° and less than 115° to the first treatment-opening surface portion 6. It is preferred if the angle β, which is between the first treatment-opening surface portion 6 and the second treatment-opening surface portion 7, is 107.57°.

An angle γ is preferably between 20° and 110°, more preferably between 80° and 100°, more preferably (exactly) 89.57°. The angle γ indicates the inclination between the inclination of the second treatment-opening surface portions 7 and the inclination of the sleeve 8 or the inserted electrode/probe 3.

The first treatment-opening surface portion 6 has the first predetermined minimum distance d1 to the electrode 3 insertable through the sleeve or through hole 8. The first predetermined minimum distance d1 is preferably between 5.66 mm and 7.66 mm, more preferably 6.66 mm. The second treatment-opening surface portion 7 has the second predetermined minimum distance d2 to the electrode 3 insertable through the sleeve or through hole 8. The second predetermined minimum distance d2 is preferably between 1 mm and 7.5 mm, more preferably 4.96 mm. It is essential that the first predetermined minimum distance d1 is greater than the second predetermined minimum distance d2. This forces a user to press application cap 1 with the distal end of the against the target tissue in such a way that the distance between the distal end of the electrode 3 and the target tissue, when an arc is generated via the first treatment-opening surface portion 6, is smaller than the distance to the target tissue via the second treatment-opening surface portion 7.

As shown in FIG. 1, the application cap 1 is configured to fix the electrode 3 to the outside of the application cap 1 in a self-retaining manner while maintaining the first predetermined minimum distance d1 and the second predetermined minimum distance d2.

FIG. 2 is a diagram illustrating the application cap 1 according to the present disclosure. FIG. 2 is a schematic representation of the application cap 1 according to the above description of FIG. 1. Accordingly, FIG. 2 shows the application cap 1 forming or defining a spatial volume 2 at its distal portion.

The treatment opening 5 is shown in FIG. 2 and is defined by a wall region/body edge 4. In FIG. 2, the first treatment-opening surface portion 6 and the second treatment-opening surface portion 7 are shown connected as a (single) treatment opening 5. For greater clarity, the reference signs 6 and 7 are omitted in FIG. 2. In FIG. 2, the treatment opening 5 is provided in the distal direction and in the downward direction of the application cap 1.

The wall region/the body edge 4, which defines or limits/borders the first treatment-opening surface portion 6, is configured in a semicircular shape in plan view. The wall region/the body edge 4, which delimits the second treatment-opening surface portion 7, is thus formed in side view by two parallel spaced crosspieces or strips, which connect to the semicircular wall region 4, forming a flowing transition.

Furthermore, it is preferred if, starting from the sleeve 8, a guide groove 12 is provided in the upper side of the application cap 1 in the longitudinal direction of the cap. The guide groove 12 is configured to guide the electrode 3 on the outside of the application cap 1 and hold it in position. In other words, the guide groove 12 serves to prevent lateral slippage of the electrode 3.

FIG. 3 is a view from proximal toward distal of the application cap 1 according to the present disclosure. FIG. 3 shows the electrode 3 lying in the guide groove 12, which projects through the sleeve 8 into the spatial volume 2 of the application cap 1.

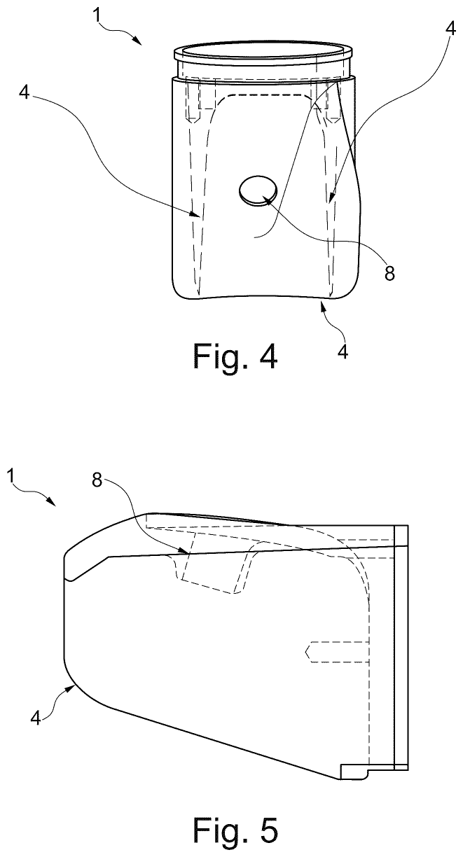

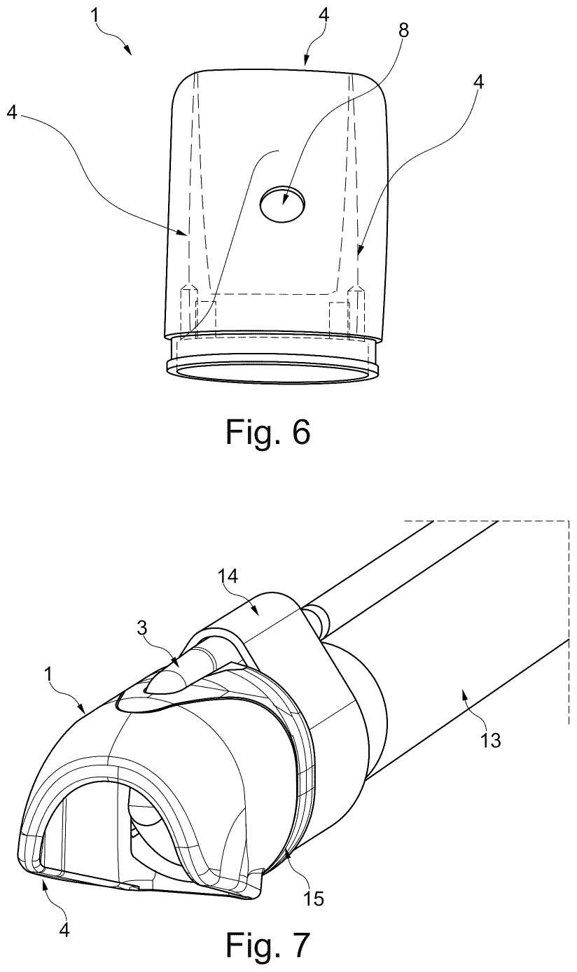

FIGS. 4 to 6 are different views of the application cap 1 with the wall region 4 and the sleeve or through hole 8. FIG. 4 is a bottom view of the application cap 1 according to the present disclosure. FIG. 5 is a side view of the application cap 1 according to the present disclosure and FIG. 6 is a top view of the application cap 1 according to the present disclosure.

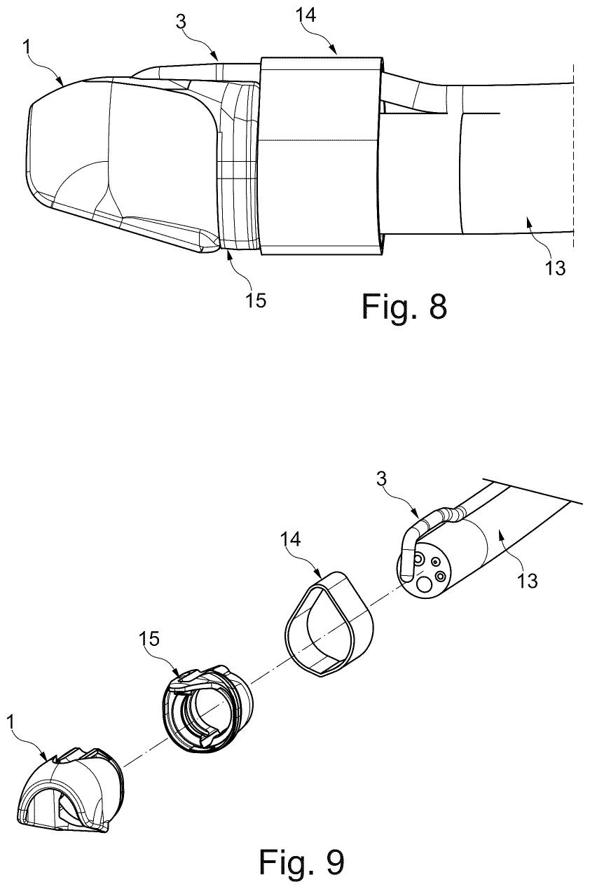

FIG. 7 is a schematic view of a plasma-surgical instrument 13 with the application cap 1 according to the present disclosure and FIG. 8 is a schematic side view of a front part of the plasma-surgical instrument 13 with the application cap 1 according to the present disclosure. Both FIG. 7 and FIG. 8 show the proximal end of the plasma-surgical instrument 13. The application cap 1 is plugged on/attached to the plasma-surgical instrument 13. The probe/electrode 3 is arranged/attached, preferably fixed, to the outside of the plasma-surgical instrument 13. The distal end of the probe/electrode 3 is inserted into the application cap 1. In order to securely fix the electrode/probe 3, a fixing ring 14 is provided, which encloses the electrode/probe 3 and the distal end of the plasma-surgical instrument 13 with the application cap 1 attached to it. In other words, the fixing ring 14 is configured according to a band, preferably in the form of an adhesive strip or a rubber band, to hold/fix the electrode/probe 3 in position.

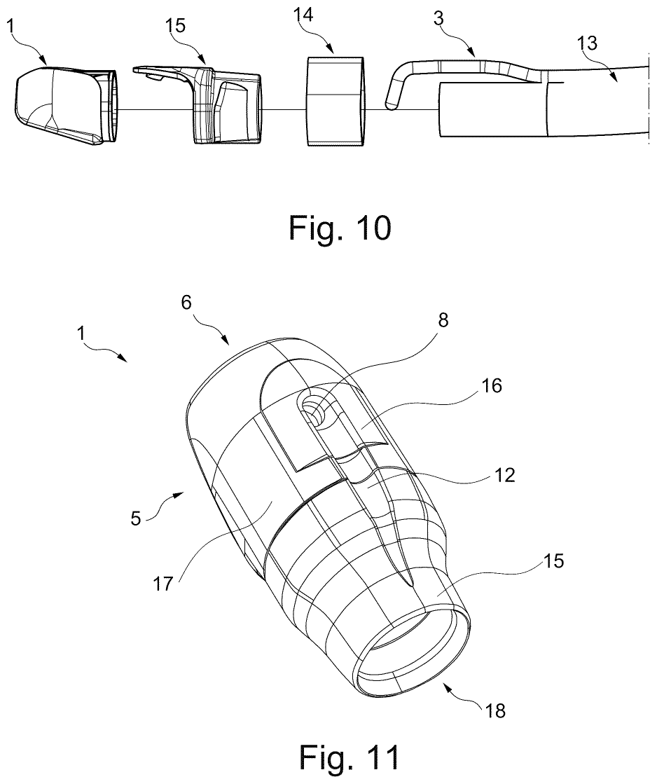

FIG. 9 is an exploded view of the application cap 1 which can be plugged onto a plasma-surgical instrument 13 and FIG. 10 is an exploded view according to FIG. 9 from the side. FIGS. 9 and 10 show, viewed from distal to proximal, the application cap 1, an adapter 15, the fixing ring 14 and the plasma-surgical instrument 13, preferably an endoscope, with the probe/electrode 3 on its outside. The probe/electrode 3 and the instrument 13 are preferably not configured in one piece. The application cap 1 and the adapter 15 are configured separately from each other in this example. The adapter 15 is configured in such a way that it can be plugged together or connected to the application cap 1, preferably via adhesive, so that alternatively the application cap 1 and the adapter 15 form a unit or become one piece.

FIG. 11 shows a second embodiment of the application cap 1. In contrast to the first embodiment, the application cap 1 has a seal 16 which is configured separately from an outer wall/circumferential wall 17 (or the adapter 15) enclosing the spatial volume 2. Preferably, the seal 16 can be inserted into a corresponding recess in the outer wall 17 and, in particular, bonded to the outer wall 17. The seal 16 forms the through hole 8 or the sleeve 8 (enclosing the through hole), through which the electrode 3 can be inserted into the spatial volume 2 from the outside. The seal 16 serves to seal between the application cap 1 and the electrode 3.

The seal 16 may preferably be made from an elastic material. In contrast, the outer wall 17 surrounding the spatial volume 2 may preferably be made from a rigid plastic. The outer wall 17 may, for example, be made from a transparent plastic.

The adapter 15 may preferably be plugged onto the application cap 1 (or may alternatively be inserted) and in particular may be bonded to the application cap 1. In the second embodiment, the adapter 15 is configured as a connecting sleeve and can preferably be configured substantially annular in cross-section. The connecting sleeve is configured from an elastic material, preferably from the same material as the seal 16. The connecting sleeve forms a push-on opening 18, with which it can be plugged onto an endoscope. Due to its elasticity, the connecting sleeve can be plugged onto endoscopes of different diameters.

As in the first embodiment, the guide groove 12, which is preferably semi-circular in cross-section, is formed in the (outer) upper side of the application cap 1 in the longitudinal direction of the cap. The guide groove 12 extends from the through hole 8 in a proximal direction. The guide groove 12 serves to guide the electrode 3, in particular to hold it laterally/sideways. In the second embodiment, the guide groove 12 is formed in portions by the seal 16, in portions by the outer wall 17 and in portions by the adapter 15.

### REFERENCE SIGN

1 application cap
2 spatial volume
3 electrode/probe
4 wall region/body edge
5 treatment opening
6 first treatment-opening surface portion
7 second treatment-opening surface portion
8 sleeve/through hole
9 longitudinal axis
10 edge
11 crosspiece
12 guide groove
13 plasma-surgical instrument
14 fixing ring
15 adapter
16 seal
17 outer wall
18 push-on opening
d1 first minimum distance
d2 second minimum distance

1-21. (canceled)

22. An application cap either for or of a plasma-surgical instrument, the application cap constituting a spacer and being configured to be coupled to a distal end of an endoscopic supply device for the plasma-surgical instrument, wherein:
the application cap forming a spatial volume on a distal portion of the application cap,
the application cap configured to maintain at least a minimum distance between a target tissue and a distal end of an electrode, which is insertable into the application cap, and
the application cap having at least one wall region which is configured to come into contact with the target tissue and in which at least one treatment opening is disposed,
the at least one treatment opening forms a first treatment-opening surface portion and a second treatment-opening surface portion, wherein the first treatment-opening surface portion is oriented in a different direction with respect to the second treatment-opening surface portion,
wherein the application cap has a laterally arranged sleeve or a laterally arranged through hole, through which the electrode can be inserted from an outside and into the spatial volume of the application cap.

23. The application cap according to claim 22, wherein the first treatment-opening surface portion and the second treatment-opening surface portion are formed contiguously to form a single treatment opening.

24. The application cap according to claim 22, wherein the first treatment-opening surface portion and/or the second treatment-opening surface portion is/are surrounded by a bearing edge which is configured to come into planar contact with the target tissue.

25. The application cap according to claim 24, wherein the bearing edge and at least one of the treatment-opening surface portions surrounded thereby lie substantially in one plane.

26. The application cap according to claim 24, wherein a first bearing edge surrounding the first treatment-opening surface portion and the first treatment-opening surface portion lie in a flattened region which extends transversely.

27. The application cap according to claim 24, wherein a second bearing edge surrounding the second treatment-opening surface portion and the second treatment-opening surface portion lie in a flattened region extending off-center as well as at a tip angle inclined distally to a longitudinal axis of the application cap.

28. The application cap according to claim 24, wherein the bearing edge encloses the respective treatment-opening surface portion at least around half of a circumference of the respective treatment-opening surface portions or completely.

29. The application cap according to claim 22, wherein the sleeve or the through hole forms a seal for sealing between the application cap and the electrode which can be inserted or is inserted through the sleeve or the through hole.

30. The application cap according to claim 22, wherein the application cap is configured substantially closed on the outside, apart from the at least one treatment opening and/or the sleeve or the through hole for the electrode.

31. The application cap according to claim 22, wherein the application cap has a circumferential wall which encloses the spatial volume and in which the sleeve or the through hole is formed.

32. The application cap according to claim 31, wherein the seal is formed as an elastic component which is separate from the circumferential wall.

33. The application cap according to claim 22, wherein the sleeve or the through hole is arranged and configured at a predetermined angle in such a way that an arc can be generated in a direction toward the first treatment-opening surface portion or toward the second treatment-opening surface portion via the electrode which can be introduced into the application cap through the sleeve or the through hole or is located therein.

34. The application cap according to claim 22, wherein the first treatment-opening surface portion is arranged or formed at the distal end, and the second treatment-opening surface portion is arranged or formed substantially opposite the sleeve or the through hole.

35. The application cap according to claim 22, wherein the second treatment-opening surface portion is inclined by more than 90° and less than 115° with respect to the first treatment-opening surface portion.

36. The application cap according to claim 22, wherein the first treatment-opening surface portion has a first predetermined minimum distance to the electrode insertable through the sleeve or the through hole and the second treatment-opening surface portion has a second predetermined minimum distance to the electrode insertable through the sleeve or the through hole, wherein the first predetermined minimum distance is greater than the second predetermined minimum distance.

37. The application cap according to claim 36, wherein the application cap is configured to self-retainingly fix the electrode to the outside of the application cap while maintaining the predetermined first minimum distance and the predetermined second minimum distance.

38. The application cap according to claim 22, wherein an edge is formed at the proximal end of the second treatment-opening surface portion, which edge is provided for scraping or scraping off the target tissue.

39. The application cap according to claim 22, wherein the sleeve or the through hole is stretchable or flexibly configured at least when the electrode is inserted.

40. The application cap according to claim 22, wherein the application cap distally has a tunnel portion with a tunnel opening forming the first treatment-opening surface portion and a tunnel bottom forming the second treatment-opening surface portion.

41. A plasmasurgical instrument comprising the application cap according to claim 22, wherein the electrode at a distal end of the instrument is adapted to generate an arc when supplied with electrical energy in a certain gas environment and at at least a predetermined minimum distance to the target tissue.
